(19) United States
(12) Patent Application Publication
(10) Pub. No.: US 2024/0212266 A1
(43) Pub. Date: Jun. 27, 2024

(54) DIRECT IMAGING METHOD FOR BLOOD CELLS FLOWING IN VASCULAR NETWORK

(71) Applicant: Korea Advanced Institute of Science and Technology, Daejeon (KR)

(72) Inventors: Wang Yuhl Oh, Daejeon (KR); Gyounghwan Kim, Daejeon (KR); Hyunsang Park, Daejeon (KR)

(21) Appl. No.: 18/534,407

(22) Filed: Dec. 8, 2023

(30) Foreign Application Priority Data
Dec. 22, 2022 (KR) 10-2022-0182249
Feb. 9, 2023 (KR) 10-2023-0017517

(51) Int. Cl. G06T 17/00; G06T 7/00

(52) U.S. Cl. CPC G06T 17/00; G06T 7/0012; G06T 2207/10056; G06T 2207/30101

(57) ABSTRACT

A direct imaging method for blood cells flowing in a vascular network is provided. The direct imaging method includes obtaining sequential images of a biological sample; generating blood cell images by removing a background signal from the sequential images; generating images without speckle noise by using spatially incoherent illumination; obtaining high-speed and high sensitivity imaging by using a high-speed camera with a high full-well capacity; generating a vascular structure image of the biological sample using the blood cell images; generating a blood cell trajectory image based on trajectories of blood cells moving along a vascular network identified in the vascular structure image through the blood cell images; and obtaining hemodynamic information based on the blood cell trajectory image.

### CROSS-REFERENCE TO RELATED APPLICATIONS

This application claims the benefit under 35 USC § 119(a) of Korean Patent Application No. 10-2022-0182249 filed on Dec. 22, 2022, and Korean Patent Application No. 10-2023-0017517 filed on Feb. 9, 2023, in the Korean Intellectual Property Office, the entire disclosures of which are incorporated herein by reference for all purposes.

### BACKGROUND

### 1. Field of Invention

The following description relates to an image processing technology for analyzing blood flow in a living body.

### 2. Description of Related Art

Research has been continuously conducted to the present on various methods to obtain information about the shape of vascular networks or blood flows. Among the methods, representative ones include confocal microscopy, optical coherence tomography (OCT), optical coherence tomography angiography (OCTA), and like. Confocal microscopy uses blood cells to which a fluorescent substance is attached or injects a contrast medium that blocks light transmission. In the case of using the blood cells to which the fluorescent substance is attached, a fluorescence signal emitted from the blood cells to which the fluorescent substance is attached is observed by a detector. In the case of injecting the contrast medium, the contrast medium flowing along a blood vessel blocks light transmission, and thus contrasting results are obtained from the detector based on the presence or absence of the contrast medium. In both cases, high-resolution vascular structure and blood flow information signals may be obtained through a confocal method by which light coming from points outside the focus is filtered and received by the detector. OCT uses equipment that may capture a three-dimensional (3D) image of the interior of a sample at a high speed in a non-contact and non-invasive manner using light interference. OCTA, which is based on OCT, identifies the presence or absence of a vascular structure and blood flow using methods that analyze a correlation between interference signals obtained at different time points. Confocal microscopy may inject a substance that may cause side effects, while OCTA may indirectly provide hemodynamic information.

### SUMMARY

An aspect of the present disclosure provides a technology for obtaining vascular structure information and blood flow information based on non-invasive and direct imaging of blood cells.

Technical aspects to be solved by the present disclosure are not limited to the aspect(s) described above, and other technical aspects not described above may be clearly understood by those skilled in the art from the description below.

According to an embodiment of the present disclosure, there is provided a direct imaging method for blood cells flowing in a vascular network, the direct imaging method including: obtaining sequential images of a biological sample: generating blood cell images by removing a background signal from the sequential images; generating a vascular structure image of the biological sample using the blood cell images: generating a blood cell trajectory image based on trajectories of blood cells moving along a vascular network identified in the vascular structure image through the blood cell images; and obtaining hemodynamic information based on the blood cell trajectory image.

In the embodiment, the obtaining of the sequential images of the biological sample may include: providing an output light by passing a light emitted from a light source through a multimode fiber; allowing the output light to be illuminated on a focal plane of the biological sample through a beam splitter and an objective lens, wherein, in response to the output light being illuminated on the focal plane of the biological sample, a scattered light is provided from the biological sample; allowing at least a portion of the scattered light to be incident on a camera through the objective lens, the beam splitter, and a tube lens; and capturing sequential images of the focal plane of the biological sample by the camera.

In the embodiment, the sequential images may comprise high-speed and high sensitivity images obtained by using a high-speed camera with a high full-well capacity and images without speckle noise generated by using spatially incoherent illumination.

In the embodiment, the generating of the blood cell images by removing the background signal from the sequential images may include: generating the blood cell images according to the following equations,

$${〈 I ( x , y , z , t ) 〉}_{{t}_{m} \pm Δ t} = \frac{1}{2 N + 1} {\sum }_{n = - N}^{N} I ( x , y , z , {t}_{m} + n Δ t ) , \\ {σ ( x , y , z , t )}_{{t}_{m} \pm Δ t} = \sqrt{\frac{1}{2 N + 1} {{\sum }_{n = - N}^{N} [ I ( x , y , z , {t}_{m} + n Δ t ) - {〈 I ( x , y , z , t ) 〉}_{{t}_{m} \pm Δ t} ]}^{2}} , \\ {I}_{bt} ( x , y , z , {t}_{m} ) = {〈 I ( x , y , z , t ) 〉}_{{t}_{m} \pm Δ t} + k \cdot {σ ( x , y , z , t )}_{{t}_{m} \pm Δ t} , and \\ {S}_{m} ( x , y , z , {t}_{m} ) = \vert Neg [ I ( x , y , z , {t}_{m} ) - {I}_{bg} ( x , y , z , {t}_{m} ) ] \vert ,$$

wherein I(x, y, z, t) denotes a sequential image obtained at a time t, wherein x and y denote horizontal coordinates of the sequential image and z denotes a coordinate in a depth direction of the sequential image; [symbol] I(x, y, z, t) [symbol] ₜ_{ₘ}_{±Δt} denotes an average image obtained by averaging (2N+1) sequential images based on a sequential image obtained centered at a time tₘ, wherein Δt denotes a capturing time interval of the sequential images; σ(x, y, z, t)ₜ_{ₘ}_{±Δt} denotes a standard deviation of the (2N+1) sequential images based on the sequential image obtained centered at the time tₘ; I_{bg}(x, y, z, tₘ) denotes a background signal of the sequential image obtained at the time tₘ; k denotes a real number; I(x, y, z, tₘ) denotes the sequential image obtained at the time tₘ: Sₘ(x, y, z, tₘ) denotes a blood cell image corresponding to the sequential image obtained at the time tₘ: and Neg[·] denotes an operator that maintains a value of an operand if it is a negative number and substitutes the value of the operand with zero (0) if it is a value other than the negative number.

In the embodiment, the generating of the vascular structure image of the biological sample using the blood cell images may include: generating the vascular structure image of the biological sample by averaging the blood cell images.

In the embodiment, the generating of the vascular structure image of the biological sample using the blood cell images may include: generating the vascular structure image of the biological sample by selecting a maximum pixel value for each pixel from the blood cell images.

In the embodiment, the generating of the blood cell trajectory image based on the trajectories of the blood cells moving along the vascular network identified in the vascular structure image through the blood cell images may include: generating the blood cell trajectory image based on trajectories of the blood cells moving along a central line of the vascular network through the blood cell images.

In the embodiment, the blood cell trajectory image may represent trajectories along which the blood cells move over time.

In the embodiment, the obtaining of the hemodynamic information based on the blood cell trajectory image may include: obtaining a blood flow velocity in the vascular network based on the blood cell trajectory image.

In the embodiment, the blood cell trajectory image may include a trajectory of at least one blood cell, and the obtaining of the blood flow velocity in the vascular network based on the blood cell trajectory image may include: calculating a slope of the trajectory of the at least one blood cell.

In the embodiment, the obtaining of the hemodynamic information based on the blood cell trajectory image may include: obtaining a flux in the vascular network based on the blood cell trajectory image.

In the embodiment, the obtaining of the flux in the vascular network based on the blood cell trajectory image may include: calculating the number of blood cells passing through the vascular network per unit time.

According to another embodiment of the present disclosure, there is provided a direct imaging method for blood cells flowing in a vascular network, the direct imaging method including: obtaining sequential image sets from different depths of a biological sample, wherein one sequential image set is provided from one of the different depths; and performing the following operations on each of the sequential image sets. The operations may include: generating blood cell images by removing a background signal from sequential images included in a corresponding sequential image set; generating a vascular structure image of the biological sample using the blood cell images; generating a blood cell trajectory image based on trajectories of blood cells moving along a vascular network identified in the vascular structure image through the blood cell images: and obtaining three-dimensional (3D) hemodynamic information based on the blood cell trajectory image.

According to embodiments described herein, there is a technical effect of acquiring vascular structure information and blood flow information based on non-invasive and direct imaging of blood cells.

### BRIEF DESCRIPTION OF THE DRAWINGS

FIG. 1 is a flowchart illustrating a direct imaging method for blood cells flowing in a vascular network of a living body according to an embodiment of the present disclosure.

FIG. 2 is a diagram illustrating a process of obtaining sequential images of a biological sample according to an embodiment of the present disclosure.

FIG. 3 illustrates a process of generating a blood cell image by removing a background signal from sequential images according to an embodiment of the present disclosure.

FIG. 4 illustrates a process of generating a blood cell trajectory image according to an embodiment of the present disclosure.

FIG. 5 illustrates a process of obtaining hemodynamic information based on a blood cell trajectory image according to an embodiment of the present disclosure.

FIG. 6 illustrates an example of a vascular structure image obtained by performing a process using a mouse brain as a biological sample according to an embodiment of the present disclosure.

FIG. 7A illustrates an example of an image displaying blood flow velocities of vascular portions calculated by applying a process based on the vascular structure image of FIG. 6 according to an embodiment of the present disclosure.

FIG. 7B illustrates an example of an image displaying flux values of vascular portions calculated by applying a process based on the vascular structure image of FIG. 6 according to an embodiment of the present disclosure.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following structural or functional descriptions of embodiments described herein are merely intended for the purpose of describing the embodiments described herein and may be implemented in various forms. However, it is to be understood that these embodiments are not construed as limited to the illustrated forms.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in the examples described herein may also be referred to as a second member, component, region, layer, or section without departing from the teachings of the examples.

Throughout the specification, when an element is described as “connected to” or “coupled to” another element, it may be directly “connected to” or “coupled to” the other component, or there may be one or more other components intervening therebetween. In contrast, when an element is described as “directly connected to” or “directly coupled to” another element, there can be no other elements intervening therebetween. Likewise, similar expressions, for example, “between” and “immediately between,” and “adjacent to” and “immediately adjacent to,” are also to be construed in the same way. As used herein, the term “and/or” includes any one and any combination of any two or more of the associated listed items.

The terminology used herein is for describing various examples only and is not to be used to limit the disclosure. The articles “a,” “an,” and “the” are intended to include the plural forms as well, unless the context clearly indicates otherwise. The terms “comprises,” “includes.” and “has” specify the presence of stated features, numbers, operations, members, elements, and/or combinations thereof, but do not preclude the presence or addition of one or more other features, numbers, operations, members, elements, and/or combinations thereof. The use of the term “may” herein with respect to an example or embodiment (for example, as to what an example or embodiment may include or implement) means that at least one example or embodiment exists where such a feature is included or implemented, while all examples are not limited thereto.

Unless otherwise defined, all terms, including technical and scientific terms, used herein have the same meaning as commonly understood by one of ordinary skill in the art to which this disclosure pertains and based on an understanding of the disclosure of the present application. Terms, such as those defined in commonly used dictionaries, are to be interpreted as having a meaning that is consistent with their meaning in the context of the relevant art and the disclosure of the present application and are not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like components and a repeated description related thereto will be omitted.

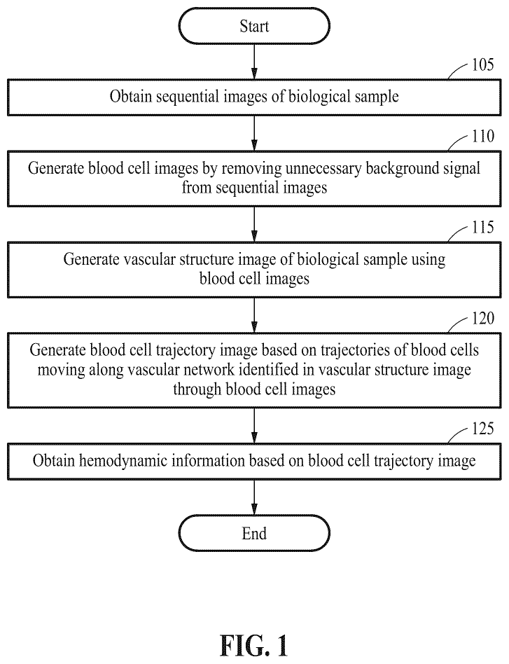

FIG. 1 is a flowchart illustrating a direct imaging method for blood cells flowing in a vascular network of a living body according to an embodiment of the present disclosure.

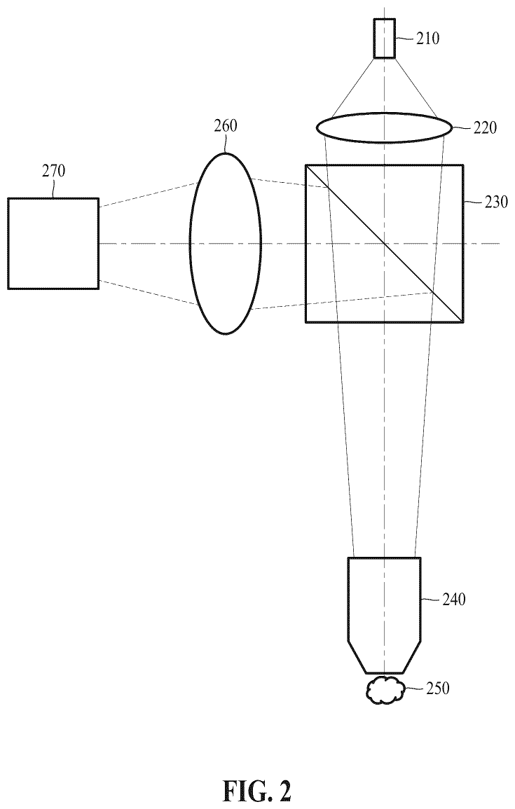

As shown in FIG. 1, the direct imaging method for moving blood cells may begin with step 105 of obtaining sequential images of a biological sample. In this step, high-speed sequential images without speckle noise may be obtained from a biological sample (e.g., a brain of a living body) by illuminating a focal plane of the biological sample through an objective lens using a spatially incoherent light (e.g., illumination) and performing imaging using a camera such as a high-speed camera. Referring to FIG. 2, which shows a process of obtaining sequential images of a biological sample, a light emitted from a light source (not shown) may first be allowed to pass through a multimode fiber 210 to provide a spatially incoherent light (e.g., illumination) for imaging without speckle noise. Subsequently, the output light may be illuminated on a focal plane of a biological sample 250 through a condenser lens 220, a beam splitter 230, and an objective lens 240. When the output light is illuminated on the focal plane of the biological sample 250, a light scattered in various directions from the biological sample 250 may be provided. At least a portion of the scattered light may be incident on a camera 270 through the objective lens 240, the beam splitter 230, and a tube lens 260. The camera 270 may then capture sequential images of the focal plane of the biological sample 250 over an imaging time T. In an embodiment, the camera 270 may be a high-speed camera capable of capturing sequential images at a high speed, for example, at a rate of one to several thousand images per second and its pixels may have large full-well capacity, for example, larger than one million photo-electrons, for high sensitivity imaging. During the capturing of sequential images of the biological sample 250 by the camera 270, the depth of the images to be captured may be adjusted by moving the biological sample 250 with respect to the objective lens 240. For example, by moving the biological sample 250 relatively far away from the objective lens 240, sequential images may be captured from a relatively small depth (shallow) of the biological sample 250. Conversely, by moving the biological sample 250 to be positioned at a relatively close distance from the objective lens 240, sequential images may be captured from a relatively great depth (deep) of the biological sample 250. In this way, three-dimensional (3D) imaging of the biological sample 250 may be performed by obtaining sequential images from different depths of the biological sample 250 while moving the biological sample 250 with respect to the objective lens 240. In the following description, a direction in which the objective lens 240 and the biological sample 250 are aligned will be referred to as a depth direction or z direction.

In step S110, blood cell images may be generated by removing a background signal from the sequential images. Equations 1 to 3 below may be used to calculate the background signal for each of the sequential images.

$${〈 I ( x , y , z , t ) 〉}_{{t}_{m} \pm Δ t} = \frac{1}{2 N + 1} {\sum }_{n = - N}^{N} I ( x , y , z , {t}_{m} + n Δ t ) \qquad [ Equation 1 ]$$

$${σ ( x , y , z , t )}_{{t}_{m} \pm Δ t} = \sqrt{\frac{1}{2 N + 1} {{\sum }_{n = - N}^{N} [ I ( x , y , z , {t}_{m} + n Δ t ) - {〈 I ( x , y , z , t ) 〉}_{{t}_{m} \pm Δ t} ]}^{2}} \qquad [ Equation 2 ]$$

$${I}_{bg} ( x , y , z , {t}_{m} ) = {〈 I ( x , y , z , t ) 〉}_{{t}_{m} \pm Δ t} + k \cdot {σ ( x , y , z , t )}_{{t}_{m} \pm Δ t} \qquad [ Equation 3 ]$$

In the equations above, I(x, y, z, t) denotes a sequential image obtained at a time t, where x and y denote horizontal coordinates of the sequential image and z denotes a coordinate in a depth direction of the sequential image. [symbol] I(x, y, z, t) [symbol] ₜ_{ₘ}_{±Δt} denotes an average image obtained by averaging (2N+1) sequential images based on a sequential image obtained centered at a time tₘ, where Δt denotes an image capturing time interval of the sequential images, σ(x, y, z, t)ₜ_{ₘ}_{±Δt} denotes a standard deviation of the (2N+1) sequential images based on the sequential image obtained centered at the time tₘ, and I_{bg}(x, y, z, tₘ) denotes a background signal of the sequential image obtained at the time tₘ. k denotes a real number.

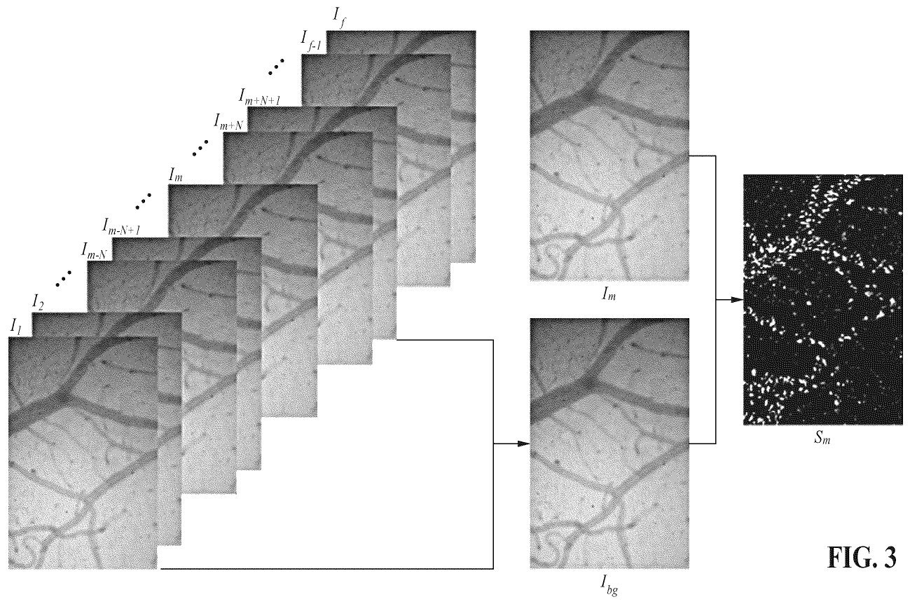

For example, as expressed in Equations 1 to 3 above, and as shown in FIG. 3, which shows a process of generating a blood cell image by removing a background signal from a sequential image, (2N+1) sequential images obtained around a time tₘ may be used to calculate a background signal I_{bg}(x, y, z, tₘ) at the time tₘ.

When the background signal I_{bg}(x, y, z, tₘ) at the time tₘ is calculated, a blood cell image Sₘ, i.e., Sₘ(x, y, z, tₘ), corresponding to the sequential image Iₘ obtained at the time tₘ may be calculated using Equation 4 below.

$${S}_{m} ( x , y , z , {t}_{m} ) = \vert Neg [ I ( x , y , z , {t}_{m} ) - {I}_{bg} ( x , y , z , {t}_{m} ) ] \vert \qquad [ Equation 4 ]$$

In Equation 4 above, I(x, y, z, tₘ) denotes an image Iₘ obtained at a time tₘ, and Sₘ(x, y, z, tₘ), denotes a blood cell image at the time tₘ. Neg[·] denotes an operator that maintains a value of an operand when it is a negative number and substitutes a value of the operand with zero (0) when it is a value other than the negative number.

In this case, the reason for obtaining the operator (e.g., Neg[·]) for a value obtained by subtracting I_{bg}(x, y, z, tₘ) from I(x, y, z, tₘ) in Equation 4 above is that, because a light absorption by blood cells is greater than that by the surrounding area and a signal intensity of pixels corresponding to the blood cells in Iₘ is lower than that of pixels corresponding to the surrounding area of the blood cells, and thus the pixels corresponding to the blood cells may have a negative value as the value obtained by subtracting I_{bg}(x, y, z, tₘ) from I(x, y, z, tₘ) and, in this case, an absolute value of the value may be used as a pixel value corresponding to the blood cells; and the pixels corresponding to the surrounding area may not have a negative value as the value obtained by subtracting I_{bg}(x, y, z, tₘ) from I(x, y, z, tₘ) and, in this case, the value may be ignored.

In step S115, a vascular structure image of the biological sample 250 may be generated using blood cell images Sₘ. In an embodiment, the vascular structure image of the biological sample 250 may be generated by averaging a sequence of the blood cell images around the time tₘ. In an embodiment, the vascular structure image of the biological sample 250 may be generated by selecting a maximum pixel value for each pixel in a sequence of the blood cell images. However, those skilled in the art may recognize that a method of generating the vascular structure image of the biological sample 250 using a sequence of blood cell images is not limited to the embodiments described above.

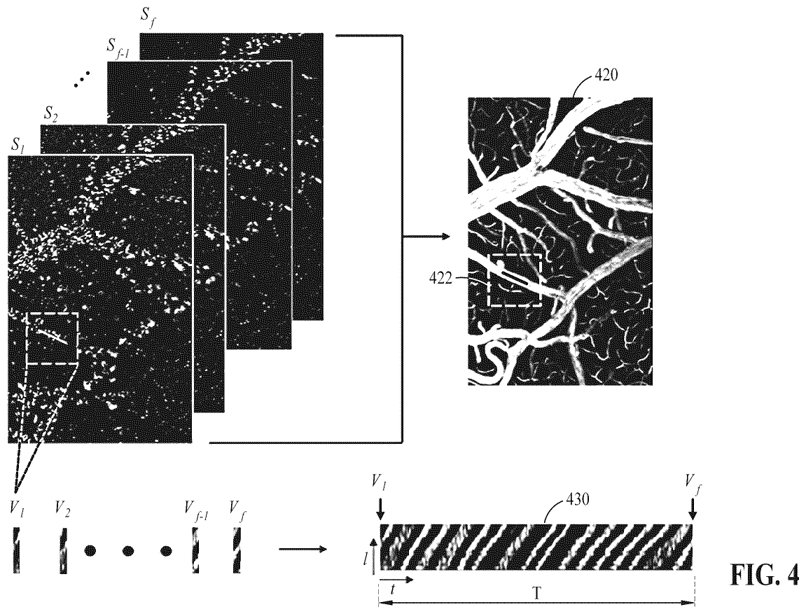

In step S120, a blood cell trajectory image may be generated based on trajectories of blood cells moving along a blood vessel (or a vascular network herein) (e.g., its central line) identified in the vascular structure image through the blood cell images Sₘ. Referring to FIG. 4, which shows a process of generating a blood cell trajectory image, blood cell images S; to S_{f} and a vascular structure image 420 generated using the blood cell images S₁ to S_{f} are shown. As shown in FIG. 4, from the blood cell images S₁ to S_{f}, partial images V₁ to V_{f} corresponding to a target vascular portion indicated in a rectangular block 422 indicated in a broken line in the vascular structure image 420 may be extracted and concatenated to generate a blood cell trajectory image 430. As shown in FIG. 4, the blood cell trajectory image 430 may have a length corresponding to each of the partial images V₁ to V_{f}, i.e., a length of the target vascular portion, in a longitudinal direction 1, and may have a time length corresponding to an imaging time T which is a total image capturing time of sequential images (captured at a certain depth) in a time direction t. The blood cell trajectory image 430 may represent the trajectories on which the blood cells move over time throughout the blood cell images S₁ to S_{f}. In general, it is assumed that blood cells move at a constant speed in a sufficiently short interval, and thus the blood cell trajectory image 430 may include diagonal lines with a consistent slope.

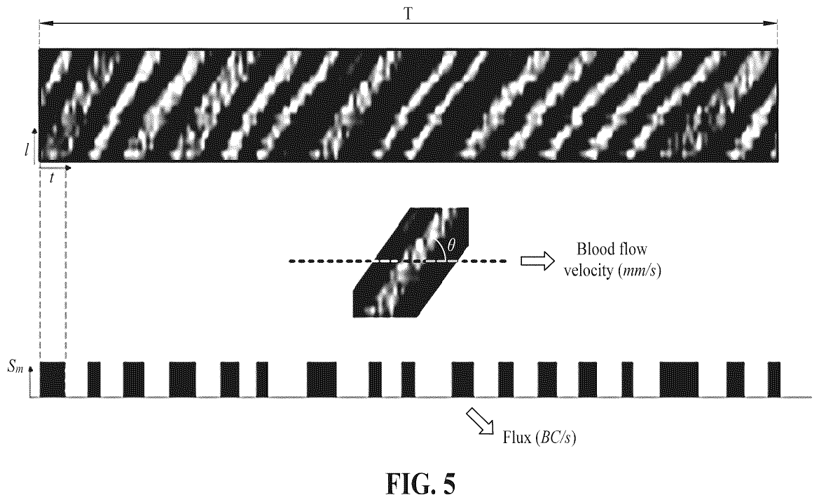

In step S125, hemodynamic information may be obtained based on the blood cell trajectory image 430. In an embodiment, the hemodynamic information may include a blood flow velocity in the target vascular portion. The blood flow velocity in the target vascular portion may be calculated by calculating a slope of a trajectory of a blood cell in the blood cell trajectory image 430 corresponding to the corresponding vascular portion, as shown in FIG. 5. In an embodiment, the hemodynamic information may include a flux in the target vascular portion. The flux in the target vascular portion may be calculated by calculating the number of blood cells passing through the corresponding vascular portion per unit time. As shown in FIG. 5, the flux in the target vascular portion may be expressed as the number of trajectory lines in the blood cell trajectory image 430 corresponding to the corresponding vascular portion, i.e., a value obtained by dividing the number of the trajectories of the blood cells by the imaging time T. Although the blood flow velocity and the flux are provided as examples of the hemodynamic information obtained in this step, those skilled in the art may recognize that hemodynamic information obtained in this step is not limited to the preceding examples.

Although it has been described above that the preceding process is used to image and analyze blood cells, which are absorbing particles whose intensity signal becomes weaker compared to those in the surrounding area, it may be recognized that the process is also used to image and analyze micro-scatterers or reflectors in a living body whose intensity signal becomes stronger compared to those in the surrounding area.

In addition, although it has been described above that sequential images are obtained from a specific depth of a biological sample and hemodynamic information is obtained based on the obtained sequential images, an embodiment in which one sequential image set is obtained from one depth and sequential image sets of different depths are provided, and as a result, 3D hemodynamic information is obtained using each of the sequential image sets may also be possible. These embodiments and variations of the embodiments should all be understood and recognized as falling within the scope of the present disclosure.

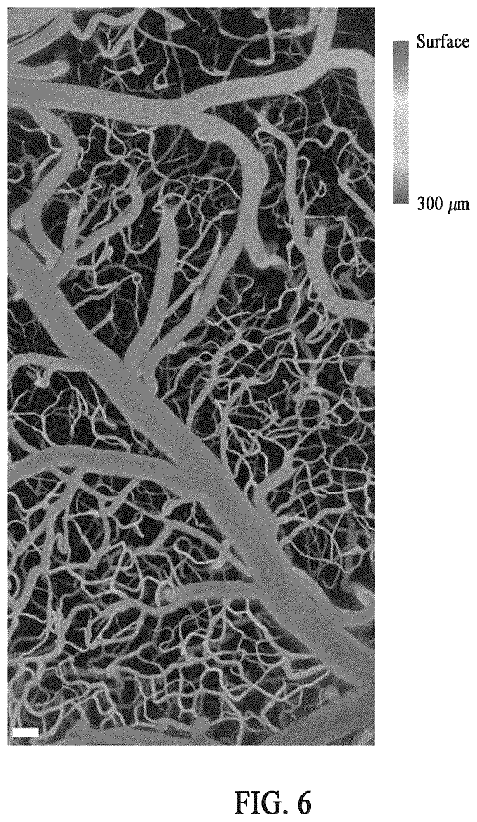

FIG. 6 illustrates an example of a vascular structure image obtained by performing a process using a mouse brain as a biological sample according to an embodiment of the present disclosure. To obtain an image shown in FIG. 6, imaging may be performed on a mouse brain while moving the mouse brain in a depth direction. From one depth, numbers of sequential images (or sequential image set) may be obtained at a frame rate of the high-speed camera (e.g. 1.450 hertz (Hz)). In addition, by analyzing numbers of sequential image sets obtained from different depths. 3D vascular structure information shown in FIG. 6 may be obtained. Referring to FIG. 6, a vascular structure obtained through imaging at a surface depth of the mouse brain, and vascular structures obtained through imaging at deeper depths (e.g., 1 to 300 μm) may be verified. As shown in the image of FIG. 6, a vascular structure of the mouse brain may be identified across all areas, including arteries, veins, and capillaries.

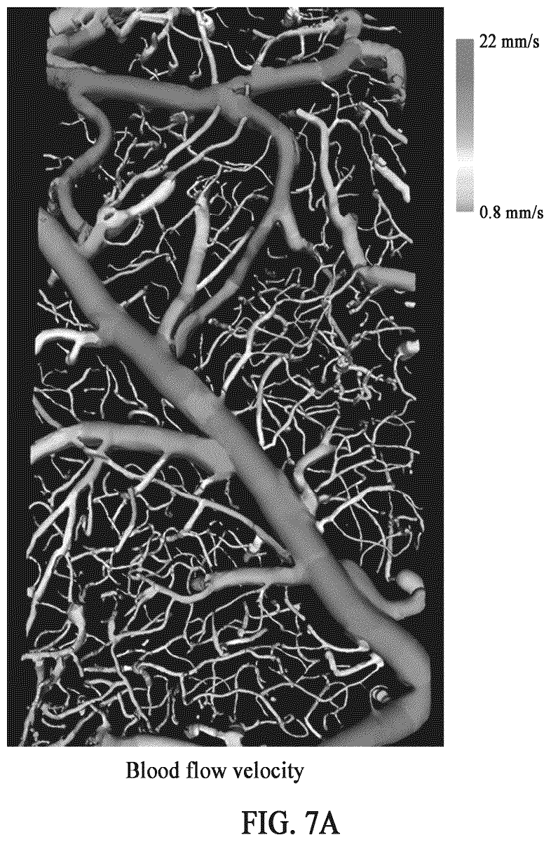

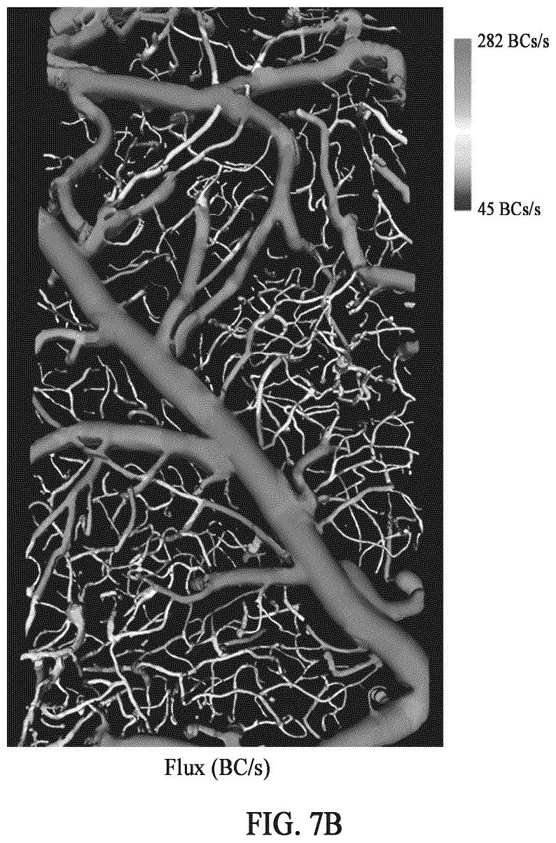

FIG. 7A illustrates an example of an image displaying blood flow velocities of vascular portions calculated by applying a process based on the vascular structure image of FIG. 6 according to an embodiment of the present disclosure. Referring to FIG. 7A, it may be verified that a blood flow velocity is relatively high in vascular portions having a relatively large blood vessel diameter.

FIG. 7B illustrates an example of an image displaying flux values of vascular portions calculated by applying a process based on the vascular structure image of FIG. 6 according to an embodiment of the present disclosure. Referring to FIG. 7B, it may also be verified that a flux value is relatively high in vascular portions having a relatively large blood vessel diameter.

The embodiments described herein may be implemented using hardware components, software components and/or combinations thereof. A processing device may be implemented using one or more general-purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such as, parallel processors.

The software may include a computer program, a piece of code, an instruction, or some combination thereof, to independently or collectively instruct or configure the processing device to operate as desired. Software and/or data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be those specially designed and constructed for the purposes of examples, or they may be of the kind well-known and available to those having skill in the computer software arts. Examples of non-transitory computer-readable media include magnetic media such as hard disks, floppy disks, and magnetic tape: optical media such as CD-ROM discs, DVDs, and/or Blue-ray discs; magneto-optical media such as optical discs: and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described hardware devices may be configured to act as one or more software modules in order to perform the operations of the above-described examples, or vice versa.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above disclosure, the scope of the disclosure may also be defined by the claims and their equivalents, and all variations within the scope of the claims and their equivalents are to be construed as being included in the disclosure.

What is claimed is:

1. A direct imaging method for blood cells flowing in a vascular network, comprising:
obtaining sequential images of a biological sample;
generating blood cell images by removing a background signal from the sequential images;
generating a vascular structure image of the biological sample using the blood cell images;
generating a blood cell trajectory image based on trajectories of blood cells moving along a vascular network identified in the vascular structure image through the blood cell images; and
obtaining hemodynamic information based on the blood cell trajectory image.

2. The direct imaging method of claim 1, wherein the obtaining of the sequential images of the biological sample comprises:
providing an output light by passing a light emitted from a light source through a multimode fiber;
allowing the output light to be illuminated on a focal plane of the biological sample through a beam splitter and an objective lens, wherein, in response to the output light being illuminated on the focal plane of the biological sample, a scattered light is provided from the biological sample;
allowing at least a portion of the scattered light to be incident on a camera through the objective lens, the beam splitter, and a tube lens; and
capturing sequential images of the focal plane of the biological sample by the camera.

3. The direct imaging method of claim 2, wherein the sequential images comprise high-speed and high sensitivity images obtained by using a high-speed camera with a high full-well capacity and images without speckle noise generated by using spatially incoherent illumination.

4. The direct imaging method of claim 1, wherein the generating of the blood cell images by removing the background signal from the sequential images comprises:
generating the blood cell images according to the following equations,
$${〈 I ( x , y , z , t ) 〉}_{{t}_{m} \pm Δ t} = \frac{1}{2 N + 1} {\sum }_{n = - N}^{N} I ( x , y , z , {t}_{m} + n Δ t ) , \\ {σ ( x , y , z , t )}_{{t}_{m} \pm Δ t} = \sqrt{\frac{1}{2 N + 1} {{\sum }_{n = - N}^{N} [ I ( x , y , z , {t}_{m} + n Δ t ) - {〈 I ( x , y , z , t ) 〉}_{{t}_{m} \pm Δ t} ]}^{2}} , \\ {I}_{bg} ( x , y , z , {t}_{m} ) = {〈 I ( x , y , z , t ) 〉}_{{t}_{m} \pm Δ t} + k \cdot {σ ( x , y , z , t )}_{{t}_{m} \pm Δ t} , and \\ {S}_{m} ( x , y , z , {t}_{m} ) = \vert Neg [ I ( x , y , z , {t}_{m} ) - {I}_{bg} ( x , y , z , {t}_{m} ) ] \vert ,$$
wherein I(x, y, z, t) denotes a sequential image obtained at a time t, wherein x and y denote horizontal coordinates of the sequential image and z denotes a coordinate in a depth direction of the sequential image, [symbol]
I(x, y, z, t) [symbol] ₜ_{ₘ}_{±Δt} denotes an average image obtained by averaging (2N+1) sequential images based on a sequential image obtained centered at a time tₘ, wherein Δt denotes a capturing time interval of the sequential images,
σ(x, y, z, t)ₜ_{ₘ}_{±Δt} denotes a standard deviation of the (2N+1) sequential images based on the sequential image obtained centered at the time tₘ,
I_{bg}(x, y, z, tₘ) denotes a background signal of the sequential image obtained at the time tₘ,
k denotes a real number,
I(x, y, z, tₘ) denotes the sequential image obtained at the time tₘ,
Sₘ(x, y, z, tₘ) denotes a blood cell image corresponding to the sequential image obtained at the time tₘ, and
Neg[·] denotes an operator that maintains a value of an operand if it is a negative number and substitutes the value of the operand with zero (0) if it is a value other than the negative number.

5. The direct imaging method of claim 1, wherein the generating of the vascular structure image of the biological sample using the blood cell images comprises:
generating the vascular structure image of the biological sample by averaging the blood cell images.

6. The direct imaging method of claim 1, wherein the generating of the vascular structure image of the biological sample using the blood cell images comprises:
generating the vascular structure image of the biological sample by selecting a maximum pixel value for each pixel from the blood cell images.

7. The direct imaging method of claim 1, wherein the generating of the blood cell trajectory image based on the trajectories of the blood cells moving along the vascular network identified in the vascular structure image through the blood cell images comprises:
generating the blood cell trajectory image based on trajectories of the blood cells moving along a central line of the vascular network through the blood cell images.

8. The direct imaging method of claim 7, wherein the blood cell trajectory image represents trajectories along which the blood cells move over time.

9. The direct imaging method of claim 7, wherein the obtaining of the hemodynamic information based on the blood cell trajectory image comprises:
obtaining a blood flow velocity in the vascular network based on the blood cell trajectory image.

10. The direct imaging method of claim 9, wherein the blood cell trajectory image comprises a trajectory of at least one blood cell,
wherein the obtaining of the blood flow velocity in the vascular network based on the blood cell trajectory image comprises:
calculating a slope of the trajectory of the at least one blood cell.

11. The direct imaging method of claim 7, wherein the obtaining of the hemodynamic information based on the blood cell trajectory image comprises:
obtaining a flux in the vascular network based on the blood cell trajectory image.

12. The direct imaging method of claim 11, wherein the obtaining of the flux in the vascular network based on the blood cell trajectory image comprises:
calculating the number of blood cells passing through the vascular network per unit time.

13. A direct imaging method for blood cells flowing in a vascular network, comprising:
obtaining sequential image sets from different depths of a biological sample, wherein one sequential image set is provided from one of the different depths; and
performing the following operations on each of the sequential image sets,
wherein the operations comprise:
in generating blood cell images by removing a background signal from sequential images comprised in a corresponding sequential image set;
generating a vascular structure image of the biological sample using the blood cell images;
generating a blood cell trajectory image based on trajectories of blood cells moving along a vascular network identified in the vascular structure image through the blood cell images; and
obtaining three-dimensional (3D) hemodynamic information based on the blood cell trajectory image.

14. A non-transitory computer-readable storage medium storing instructions that, when executed by a processor, cause the processor to perform the direct imaging method of any one of claim 1.
